(19) United States
(12) Patent Application Publication
(10) Pub. No.: US 2004/0063630 A1
(43) Pub. Date: Apr. 1, 2004

(54) Treatment of congestive heart failure with natriuretic peptide and a diuretic

(75) Inventor: George F. Schreiner, Los Altos, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Scios, Inc.

(21) Appl. No.: 10/390,545

(22) Filed: Mar. 18, 2003

Related U.S. Application Data
(60) Provisional application No. 60/364,638, filed on Mar. 18, 2002.

(51) Int. Cl. A61K 38/17

(52) U.S. Cl. 514/12

(57) ABSTRACT

Compositions and methods are disclosed for administration of synergistic levels of a diuretic and a natriuretic peptide, especially a recombinant form of human BNP, or hBNP. These compositions and methods are useful for the treatment of congestive heart failure.

### BACKGROUND OF THE INVENTION

1. Field of the Invention

The invention relates to natriuretic peptide and congestive heart failure. Specifically, this invention is directed to compositions and methods comprising natriuretic peptide, or derivatives of this compound, and a diuretic to relieve pulmonary vascular congestion and promote diuresis. These compositions and methods are useful for achieving the combined effect of treating congestive heart failure and promoting diuresis where it is desired to obtain enhanced diuresis (including diuresis with reduced side-effects) relative to the quality thereof that would otherwise be observed upon administration of a natriuretic peptide, alone, or administration of a diuretic agent, alone.

2. Background Art

A diuretic, such as furosemide, is frequently administered to patients with acutely decompensated heart failure to relieve pulmonary vascular congestion and promote diuresis. The efficacy of furosemide is based on its ability to markedly enhance sodium excretion and decrease intravascular volume despite the presence of decreased renal perfusion pressure. However, furosemide is associated with a variety of deleterious effects and, by itself, may not consistently cause diuresis or natriuresis. The administration of intravenous furosemide causes a slight increase in mean arterial pressure and systemic vascular resistance and a decrease in cardiac output before the onset of diuresis in patients with heart failure (Francis, G. S., et al, Ann Intern Med 103:1-6 (1985)). This results in a decrease of renal blood flow (Mudge, G. H., et al., Am. Jour. Physiol. 228(5):1304-1312 (1975); Stein, J. H., et al., J. Lab. Clin. Med. 79:995-1003 (1972)) and a decrease in glomerular filtration rate (Fett, D. L., et al., J. Am. Soc. Nephro.l. 4:162-167 (1993)). These effects are related to the activation of the renin-angiotensin-aldosterone (RAA) system (Ikram, H., et al., Clin. Sci. 59:443-449 (1980); Schaer, G., et al., Am. J. Cardiol. 51:1635-1638 (1983); Bayliss, J., et al., Br. Heart J. 57:17-22 (1987)). In addition, the plasma norepinephrine level increases acutely after administration of intravenous furosemide. Chronic use of furosemide further aggravates symptomatic nervous system (SNS) and RAA activation due to relative reductions in intravascular volume. The net result of these changes stimulates the kidney to increase sodium reabsorption proximal to the loop of Henle, where furosemide acts, resulting in a state of clinical resistance to furosemide in the context of congestive heart failure (D. Ellison, Cardiology 96:132-43 (2001).

Brain natriuretic peptides (BNP) in pharmacological doses has been found to have favorable effects on the hemodynamic profile of patients with heart failure, producing a fall in systemic vascular resistance and a mild reduction in arterial pressure (Colucci, W. S., et al., N. Engl. J. Med. 343:246-253 (2000)). The neuroendocrinologic alterations seen after the administration of BNP include a decrease in aldosterone levels and a mild decrease in plasma renin activity (McGregor, A., et al., J. Clin. Endo. Metab. 70:1103-1107 (1990); Holmes, S. J., et al., J. Clin. Endo. Metab. 76: 91-96 (1993); Yoshimura, M., et al., Circulation 84:1581-1588 (1991)). As a result, BNP inhibits the antinatriuretic effect of angiotensin II and aldosterone on the proximal and distal convoluted tubules. BNP increases distal sodium delivery and decreases proximal and distal tubular sodium reabsorption. BNP maintains glomerular filtration rate and has modest diuretic properties with increases in urinary sodium and volume (Marcus, L. S., et al., Circulation 94:3184-3189 (1996)).

### SUMMARY OF THE INVENTION

Brain natriuretic peptides (e.g., BNP), where given in association with diuretics such as furosemide, reduce or eliminate the unfavorable effects of the diuretic on RAA, SNS and vasculature, while maintaining beneficial effects of the diuretic. Accordingly, the present invention is directed to the need to relieve diuretic-associated side-effects in patients with congestive heart failure. Recognizing that the administration of diuretics to relieve pulmonary vascular congestion and promote diuresis in these patients can cause deleterious side-effects, the present invention involves combined administration of natriuretic peptide and a diuretic, to unexpectedly reduce the acute vasoconstrictor and neuroendocrinologic properties of diuretics while augmenting the diuretic and natriuretic effects. Accordingly, the present invention is directed to compositions comprising efficacious amounts of natriuretic peptide and a diuretic, especially BNP and/or ANP and most preferably human BNP. Further, the invention is directed to methods for reducing diuretic-associated side-effects in an animal, such methods comprising administration of effective amounts of BNP or an active derivative thereof to such animal in combination with or subsequent to administration of a diuretic agent. These compositions and methods are useful in the treatment of congestive heart failure, particularly to relieve pulmonary vascular congestion where promotion of diuresis is also desired with reduction in side effects typically seen with conventional diuretic agents.

Accordingly, in a first embodiment, the invention is directed to a composition of method for the administration of natriuretic peptide to a patient who has been diagnosed as having congestive heart failure and who is in need of management of ongoing risk of heart failure, such method comprising administration of a therapeutically effective dose of natriuretic peptide, to said patient, in combination with a diuretic.

In a further embodiment, the natriuretic peptide is atrial natriuretic peptide (ANP) or B-type natriuretic peptide (BNP).

In a further embodiment, such natriuretic peptide is a human natriuretic peptide.

In a further embodiment, the human natriuretic peptide is recombinant or synthetic human B-type natriuretic peptide (nesiritide).

### DETAILED DESCRIPTION OF THE INVENTION

Definitions

The term “ameliorate” denotes a lessening of an effect. To ameliorate a condition or disease refers to a lessening of the symptoms of the condition or disease.

An “individual” is a vertebrate, preferably a mammal, more preferably a human.

“Mammal” refers to any animal classified as a mammal, including humans, domestic and farm animals, and zoo, sport, or pet animals, such as, for example, horses, sheep, cows, pigs, dogs, cats, etc. Preferably, the mammal is human.

A “therapeutically effective amount” or an “effective amount” is an amount sufficient to effect beneficial or desired results. An effective amount can be provided in one dose. Alternatively, the effective amount can be provided in multiple doses for a desired period of time, such multiple doses being cumulatively sufficient to effect the beneficial or desired result but each of such multiple doses being at an individual level that may or may not be effective had it been administered by itself.

“Administration” means any manner of providing a desired agent to a subject or patient. Administration “in combination with” one or more further therapeutic agents means any manner with provides for the beneficial effects of the administration of both agents, including simultaneous (concurrent) administration and consecutive administration in any order.

The term “modulate” means to control in a predictable fashion, either by increasing or by decreasing the targeted parameter, as indicated from the context.

A “treatment” is an approach for obtaining a beneficial or desired result, especially a clinical result, especially the administration of an agent to a subject for purposes which may include prophylaxis, amelioration, prevention or cure of an undesired physiological condition or disease. Such treatment need not necessarily completely ameliorate the condition or disorder. For purposes of this invention, beneficial or desired clinical results include, but are not limited to, alleviation of symptoms, diminishment of extent of tissue injury or disease, stabilized (i.e., not worsening) state of tissue injury or disease, delay or slowing of the progression or tissue injury or disease, amelioration or palliation of an undesired physiological condition or disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

“Carriers” as used herein include pharmaceutically acceptable carriers, excipients, or stabilizers which are nontoxic to the cell or mammal being exposed thereto at the dosages and concentrations employed.

The term “pharmaceutically acceptable salt” as used herein refers to salt forms of a substance that are substantially non-toxic to living organisms. Typical pharmaceutically acceptable salts include those salts prepared by reaction of a desired agent, such as a desired form of natriuretic peptide, with a pharmaceutically acceptable mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of pharmaceutically acceptable salts formed from such acids are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma.-hydroxybutyrate, glycollate, tartrate, methanesulfonate, propanesulfonate, naphthalene-1-sulfonate, naphthalene-2-sulfonate, mandelate, mesylate, and the like. Preferred pharmaceutically acceptable acid addition salts are those formed with mineral acids such as hydrochloric acid and hydrobromic acid, and those formed with organic acids such as maleic acid and methanesulfonic acid.

Salts of amine groups may also comprise quaternary ammonium salts in which the amino nitrogen carries a suitable organic group such as an alkyl, alkenyl, alkynyl, or aralkyl moiety.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is not of a critical nature, so long as the salt as a whole is pharmacologically acceptable and as long as the counterion does not contribute undesired qualities to the salt as a whole.

The term “essentially free of contaminants” refers to a substance that is purified to a degree such that the substance contains no, or acceptable levels of, undesired or unnecessary substances that arose form, or had been present during, the in vitro or in vivo synthesis of the desired substance.

Congestive Heart Failure

Congestive heart failure (CHF; cardiac failure) is a condition in which weakened heart function exists together with a build-up of body fluid. Cardiac failure often occurs when cardiac output is insufficient to meet metabolic demands of the body, or when the heart cannot meet the demands of operating at increased levels of filling/diastolic pressure. Therapy involves not only support of the weakened heart function but also treatment to counteract the build up of the body fluid.

Congestive heart failure may be caused by many forms of heart disease. Common causes of congestive heart failure include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis). Each of these disease processes can lead to congestive heart failure by reducing the strength of the heart muscle contraction, by limiting the ability of the heart's pumping chambers to fill with blood due to mechanical problems or impaired diastolic relaxation, or by filling the heart's chambers with too much blood.

Advanced congestive heart failure (CHF) includes both acute and chronic presentations. Patients presenting with acute decompensated CHF usually have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries result in an acute rise in the left ventricular (LV) filing pressures. The rise in the LV filing pressures results in pulmonary edema and dyspnea. The treatment of patients with acute decompensated CHF focuses on treating the reason behind the myocardial injury. In addition, the heart's function is supported by treatments to reduce LV filling pressures and to improve cardiac performance.

Patients with chronic decompensated heart failure often have symptoms of volume overload and/or low cardiac output—but do not appear to be in a volume overloaded state. Such patients thus have a chronic LV systolic dysfunction. The treatment strategy for such patients is not clear. Typical treatments may include intravenous inotropic therapy, an LV mechanical assist device or even cardiac transplantation.

The present invention comprises compositions and methods to treat patients with congestive heart failure by administration of a diuretic and an effective amount of natriuretic peptide.

The compositions and methods of the invention are based on the inventors' discovery that natriuretic peptide, and especially, BNP reduces or eliminates diuretic-associated alterations in renal blood flow, decreases in glomerular filtration rate, acute increases in plasma norepinephrine levels, and aggravation of the SNS after chronic use. The inventors realized that BNP, in pharmacological doses, decreases systemic vascular resistance and arterial pressure in patients with heart failure. The inventors also realized that BNP decreases aldosterone levels and plasma renin activity thereby inhibiting the antinatriuretic effect of angiotensin II and aldosterone on the proximal and distal convoluted tubules. Brain natriuretic peptides (BNP), when given in association with furosemide, may reduce or eliminate the unfavorable effects of RAA, SNS and vasculature, while augmenting the beneficial effects of furosemide.

The active ingredient according to the present invention is a natriuretic peptide such as, for example, atrial natriuretic peptide (ANP), brain natriuretic peptide (BNP or B-type natriuretic peptide) and C-type natriuretic peptide (CNP). Of them, ANP and BNP are preferred, and BNP is the most preferred. Sequences of various forms of natriuretic peptide are provided in (U.S. Patent Application Publication No. 20010027181A1, incorporated by reference herein).

Specific examples of ANPs that can be used in the methods of the invention include: human ANP (human atrial natriuretic peptide; hANP, Kangawa et al., Biochem. Biophys. Res. Commun., Vol. 118, p. 131, 1984) (Seq. ID No. 1) or rat ANP (Kangawa et al., Biochem. Biophys. Res. Commun., Vol. 121, p. 585, 1984). Such ANPs comprise 28 amino acids. Such ANPs may be administered as a peptide having a ring structure of ANP (formation of a disulfide bond based on Cys), and a C-terminal portion succeeding the ring structure. An example of such a peptide is a peptide having amino acid residues at the 7-position to the 28-position of ANP is provided in U.S. Patent Application Publication No. 20010027181A1. Another example is frog ANP. Of them, human ANP (hANP), and especially recombinant hANP is particularly preferred.

Specific examples of BNPs that can be used in the methods of the invention include human BNP (hBNP; SEQ ID NO:1). Human BNP comprises 32 amino acids and involves the formation of a disulfide bond, like the above-described ANP (Sudoh et al., Biochem. Biophys. Res. Commun., Vol. 159, p. 1420, 1989). See also, U.S. Pat. Nos. 5,114,923, 5,674,710, 5,674,710, 5,948,761, each of which is hereby incorporated by reference. Various BNP's of the origin other than human, such as pig BNP and rat BNP, are also known, and can be used similarly. A further example is chicken BNP.

Specific examples of CNPs that can be used in the methods of the invention include pig CNP. Pig CNP comprises 22 amino acids and involves the formation of a disulfide bond, like the above-described ANP and BNP (Sudoh et al., Biochem. Biophys. Res. Commun., Vol. 168, p. 863, 1990) (human and rat also have the same amino acid sequence), chicken CNP (Arimura et al., Biochem. Biophys. Res. Commun., Vol. 174, p. 142, 1991). Frog CNP (Yoshihara et al., Biochem. Biophys. Res. Commun., Vol. 173, p. 591, 1990) can also be used.

Furthermore, any person skilled in the art can apply modification, such as deletion, substitution, addition or insertion, and/or chemical modification to amino acid residues in the amino acid sequence of a known natriuretic peptide (e.g., the aforementioned human ANP; hANP), as desired, by a known method. One skilled in the art can confirm that the resulting compound is a compound which has the activity of acting on a receptor of the starting ANP or BNP or CNP. Derivatives having this activity, therefore, are included in the substance as an active ingredient which is administered to a patient in accordance with the method of the present invention.

A substance that activates the patient's natriuretic peptide receptor could also be used in the compositions of the invention in place of, or in addition to, one or more of the natriuretic peptides discussed above. Such substances should be capable of acting on a natriuretic peptide receptor to increase intracellular cGMP production. Such substances may be non-peptide compounds.

In the compositions of the invention, the natriuretic peptide is preferably provided as a free (non-salt) form, or as a pharmaceutically acceptable salt. A salt with an inorganic acid preferably includes salts with hydrochloric acid, sulfuric acid, and phosphoric acid. The salt with an organic acid thus may, preferably be, for example, acid addition salts with formic acid, acetic acid, butyric acid, succinic acid, and citric acid. The salt is preferably in the form of a metal salt with sodium, potassium, lithium or calcium, or a salt with an organic base.

To produce compositions for infusion, carriers or additives can be added to provide a desired stability or property to the composition. Examples of such carriers and additives include: (1) tonicity agents such as sodium chloride, D-mannitol, and D-sorbitol, (2) pH regulators such as hydrochloric acid and citric acid, (3) buffering agents such as sodium citrate, sodium acetate, and boric acid, and (4) soothing agents such as procaine hydrochloride; as well as stabilizers, and surface active agents. Often the physiologically acceptable carrier is an aqueous pH buffered solution. Examples of physiologically acceptable carriers include buffers composed of phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

In consideration of the stability, etc. of the active natriuretic peptide ingredient, it can be selected whether the active ingredient should be formed into a preparation to be used after dissolution or suspension when required, or into a liquid preparation.

The Natriuretic peptides and derivatives thereof that are capable of activating the natriuretic peptide receptor as discussed above can be administered by any method which results in deliverance of efficacious levels of such peptides such that amelioration of the diuretic-associated side-effect occurs. For example, the composition that contains the active natriuretic peptide substance may be administered parenterally, orally, or topically, in dosage unit formulations containing conventional nontoxic pharmaceutically acceptable carriers, adjuvants and vehicles as desired. The term parenteral as used herein includes subcutaneous, intravenous, intraarterial injection or infusion techniques, without limitation. The term “topically” encompasses administration rectally and by inhalation spray (aerosol), as well as by the more common routes of the skin and the mucous membrane of the mouth and nose.

Total daily doses of the compounds of this invention administered to a host in single or divided doses may be in amounts. Typically, a recommended dose of 2 μg/kg bolus of a natriuretic peptide such as Natrecor® has been used and this dose has been followed by a continuous infusion at a dose of 0.01 μg/kg/min. However, a bolus that is more or less than 2 μg/kg coupled with continuous infusion at more or less than the previous dose of 0.01 μg/kg/min may also be efficacious.

Dosage unit composition may contain such amounts of such submultiples thereof as may be used to make up the daily doses. It will be understood however, that the specific dose level for any particular patient will depend upon a variety of factors including the body weight, general health, sex, diet, time and routine of administration, rates of absorption and excretion, combination with other drugs and the severity of the particular diuretic-associated side-effect being treated.

Injectable preparations, such as oleaginous solutions, suspensions or emulsions, may be formulated according to known art, using suitable dispersing or wetting agents and suspending agents, as needed. When the active compounds are in water-soluble form, for example, in the form of water soluble salts, the sterile injectable preparation may employ a nontoxic parenterally acceptable diluent or solvent as, for example, sterile nonpyrogenic water or 1,3-butanediol. Among the other acceptable vehicles and solvents that may be employed are 5% dextrose injection. Ringer's injection and isotonic sodium chloride injection (as described in the USP/NF). When the active compounds are in a non-water soluble form, sterile, appropriate oily suspensions containing suitable lipophilic solvents or vehicles, such as fatty oil, for example, sesame oil, or synthetic fatty acid esters, for example, ethyl oleate or triglycerides, are used. Alternatively, aqueous injection suspensions which contain substances which increase the viscosity, for example, sodium carboxymethyl cellulose, sorbitol, and/or dextran, and optionally also contain stabilizers may be used.

The pharmaceutical preparations of the present invention are manufactured in a manner which is in itself known, for example, by means of conventional mixing, granulating, dragee-making, dissolving, or lyophilizing processes. Thus, pharmaceutical preparations for oral use can be obtained by combining the active compounds with solid excipients, optionally granulating a resulting mixture and processing the mixture or granules, after adding suitable auxiliaries, if desired or necessary, to give tablets of dragee cores.

Suitable excipients are, in particular, fillers such as sugars, for example lactose or sucrose, mannitol or sorbitol, cellulose preparations and/or calcium hydrogen phosphate, as well as binders, such as starch, pastes, using, for example, maize starch, wheat starch, rice starch, or potato starch, gelatine, tragacanth, methylcellulose, hydroxypropylmethyl cellulose, sodium carboxymethyl cellulose, and/or, if desired disintegrating agents, such as the above-mentioned starches, and also carboxymethyl starch, cross-linked polyvinyl pyrrolidone, agar or alginic acid or a salt thereof, such as sodium alginate. Auxiliaries are, above all, flow-regulating agents and lubricants, for example, silica, talc, stearic acid or salts thereof, such as magnesium stearate or calcium stearate, with suitable coating, which, if desired, are resistant to gastric juices and for this purpose, inter alia concentrated sugar solutions, which optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetyl cellulose phthalate or hydroxypropylmethyl cellulose phthalate, are used. Dyestuffs or pigments may be added to the tablets or dragee coatings, for example, for identification or in order to characterize different combinations of active compound doses.

Other pharmaceutical preparations which can be used orally are push-fit capsules made of gelatin, as well as soft, sealed capsules made of gelatin and a plasticizer such as glycerol or sorbitol. The push-fit capsules can contain the active compounds in the form of granules, for example, mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin or liquid polyethylene glycols, it also being possible to add stabilizers.

Suppositories for rectal administration of the compound of this invention can be prepared by mixing the drug with suitable suppository bases such as a nonirritaing excipient, for example, cocoa butter, natural or synthetic triglycerides, paraffin hydrocarbons, polyethylene glycols, or higher alkanols, and especially bases which are solid at ordinary temperature but liquid at body temperature and which therefore melt in the rectum and release drug. In addition, it is possible to use gelatin rectal capsules which consist of a combination of the active compounds with a base; possible base materials are, for example, liquid triglycerides, polyethylene glycols, or paraffin hydrocarbons.

Solid dosage forms for oral administration include capsules, tablets, pills, troches, lozenges, powders and granules. In such solid dosage forms, the active compound may be admixed with at least on inert diluent such as sucrose, lactose or starch. Such dosage forms may also comprise, as is normal practice, pharmaceutical adjuvant substances, e.g., stearate lubricating agents. Solid oral preparations can also be prepared with enteric or other coatings which modulate release of the active ingredients.

Liquid dosage forms for oral administration include pharmaceutically acceptable emulsions, solutions, suspensions, syrups and elixirs containing inert nontoxic diluents commonly used in the art, such as water and alcohol. Such compositions may also comprise adjuvants, such as wetting agents, emulsifying, suspending, sweetening, flavoring and perfuming agents.

The compositions of the present invention, in and of themselves, find utility in the control of diuretic-associated side-effects for the treatment of congestive heart failure. In intravenous dosage form, the compositions of the present invention have a sufficiently rapid onset of action to be useful in the management of diuretic-associated side-effects.

The pharmaceutically active compositions that provide the active natriuretic peptide are preferably administered to the patient who is in need of the same in the form of an injection. Such injections can be, for example, intravenous, intramuscular, subcutaneous, intradermal, intrasternal, intraperitoneal or intra-articular. Most preferably, the compositions are provided in the form of an infusion, and especially, an intravenous infusion.

The infusion can be administered for any effective period of time, for example, 1, 2, 3, 4, 5, 6, 7, 8, 9 or 10 hours, or a desired period of time in between. In a further embodiment, infusions for greater than 10 hours are performed. In a preferred embodiment, a patient in need of such treatment is infused for 4-6 hours. Preferably such infusion is continuous although two infusions of shorter duration following one right after the other may be used.

The infusion rate may be any that is tolerated by the patient. In a preferred embodiment, the infusion rate is about 0.00125 μg/kg/min to about 0.01 μg/kg/min. In a further preferred embodiment, an infusion rate of about 0.005 μg/kg/min is used.

The infusion rate should be sufficient to provide a therapeutically effective amount of the natriuretic peptide during the infusion period or treatment protocol but without compromising patient safety.

The infusion rate should be sufficient to provide a therapeutically effective amount of the natriuretic peptide during the infusion period or treatment protocol but without compromising patient safety.

The optimal volume of the infusion and amount of the active natriuretic peptide will vary by body weight. For example, a 30 kg (66 pound) patient might first be given a 2.5 ml bolus of 0.5 μg/ml of the natriuretic peptide composition infused at 0.8 ml/hr so as to provide for 0.0025 μg/kg/min, followed by a minimum bolus of 0.25 μg/kg in 1.3 mL infused at 0.4 mL/hr and 0.00125 μg/kg/min to a maximum bolus of 1.0 μg/kg in 5.0 mL infused at a maximal rate of 1.5 mL/hr to provide 0.005 μg/kg/min.

For another example, a 30 kg (66 pound) patient might first be given a 5.0 ml bolus of 1.0 μg/ml of the natriuretic peptide composition infused at 1.5 m/hr so as to provide for 0.005 μg/kg/min, followed by a minimum bolus of 0.5 μg/kg in 2.5 mL infused at 0.8 mL/hr and 0.0024 μg/kg/min.

In contrast, a 175 kg (386 pound) patient might first be given a 14.6 ml bolus of 0.5 μg/ml of the natriuretic peptide composition infused at 4.4 ml/hr so as to provide for 0.0025 μg/kg/min, followed by a minimum bolus of 0.25 μg/kg in 7.3 mL infused at 2.2 mL/hr and 0.00124 μg/kg/min to a maximum bolus of 1.0 μg/kg in 29.2 mL infused at a maximal rate of 8.8 mL/hr to provide 0.005 μg/kg/min.

Alternatively, a 175 kg (386 pound) patient might first be given a 29.2 ml bolus of 1.0 μg/ml of the natriuretic peptide composition infused at 8.8 ml/hr so as to provide for 0.005 μg/kg/min, followed by a minimum bolus of 0.5 μg/kg in 14.6 mL infused at 4.4 mL/hr and 0.0025 μg/kg/min.

In one embodiment, a separate initial bolus of a preparation that contains natriuretic peptide is administered to the patient immediately prior to a subsequent infusion. Such a bolus preferably provides from about 0.25, 0.5, 0.75, 1.0, 1.25, 1.5 or 1.75 μg/kg natriuretic peptide.

Any diuretic that is available for administration to a patient in need of the same may be used in the invention. Examples of such diuretics include, but are not limited to loop diuretics, thiazide diuretics, and potassium sparing (K-sparing) diuretics.

Loop diuretics are preferred for use with CHF patients. Loop diuretics useful in the method of the invention include furosemide (Lasix®), bumetanide (Bumex®) and torsemide (Demadex®). A preferred dose of furosemide is 20-80 mg qd-bid. A preferred dose of bumetanide is 0.5-2.0 mg qd-bid. A preferred dose of torsemide is 10-40 mg qd-bid.

Thiazide and Thiazide-like diuretics that are useful in the methods of the invention include chlorothiazide, hydrochlorothiazide (HCTZ), benzthiazide, cyclothiazide, indapamide, chlorthalidone, bendroflumethizide and metolzone.

Potassium sparing diuretics that are useful in the methods of the invention include amiloride, triamterene and spironolacton.

The preferred embodiments utilize Natrecor® as the natriuretic peptide. Natrecor® is a proprietary name for a recombinant form of human B-type natriuretic peptide (hBNP), also known as brain natriuretic peptide. It is identical to the endogenous hormone that is produced primarily by the ventricular myocardium. However, it is to be understood that the invention is not restricted to Natrecor®. The invention provides a method for the use of any natriuretic peptide. The combined administration of natriuretic peptides for treatment of congestive heart failure with administration of diuretics to promote diuresis results in an unexpected synergistic effect such that the overall improvement of a subject's diuresis (including a reduced extent of diuretic side effects) is unexpectedly better than one would have expected upon administration of the natriuretic peptide alone or the diuretic agent alone.

The effects of the natruiretic peptide/diuretic combination, for example, the Natrecor®/furosemide combination, on renal function can be determined by examining one or more parameters including urine volume, glomerular filtration rate (GRF), renal blood flow and natriuresis compared with either drug when given alone.

Those skilled in the art will appreciate that dosages of both the natriuretic peptide and the diuretic can be adjusted to achieve an optimal synergistic effect for each patient, as desired. Other suitable modifications and adaptations of the variety of conditions and parameters normally encountered in clinical therapy and which are obvious to those skilled in the art are within the spirit and scope of this invention.

What is claimed is:

1. A method for treating congestive heart failure in a mammal comprising administering a therapeutically effective dose of a natriuretic peptide in combination with a diuretic agent to said mammal.

2. The method of claim 1, wherein said dose is an adjunct to oral therapy.

3. The method of claim 1, wherein the said mammal is in a compensated state of congestive heart failure.

4. The method of claim 1, wherein the said mammal is in a decompensated state of congestive heart failure.

5. The method of claim 1, wherein said natriuretic peptide is selected from the group consisting of an atrial natriuretic peptide (ANP), a B-type natriuretic peptide (BNP) and a C-type natriuretic peptide (CNP).

6. The method of claim 5, wherein the said natriuretic peptide is B-type natriuretic peptide.

7. The method of claim 6, wherein said B-type natriuretic peptide (BNP) is selected from the group consisting of human BNP, pig BNP, rat BNP, and chicken BNP.

8. The method of claim 6, wherein said natriuretic peptide is a human B-type natriuretic peptide.

9. The method of claim 6, wherein said natriuretic peptide is a recombinant B-type natriuretic peptide.

10. The method of claim 5, wherein said natriuretic peptide is atrial natriuretic peptide (ANP).

11. The method of claim 10, wherein said atrial natriuretic peptide (ANP) is selected from the group consisting of frog ANP and human ANP.

12. The method of claim 10, wherein said atrial natriuretic peptide (ANP) is human ANP.

13. The method of claim 10, wherein said atrial natriuretic peptide (ANP) is recombinant ANP.

14. The method of claim 5, wherein said natriuretic peptide is C-type natriuretic peptide (CNP).

15. The method of claim 14, wherein said C-type natriuretic peptide (CNP) is selected from the group consisting of chicken CNP, rat CNP and human CNP.

16. The method of claim 15, wherein said C-type natriuretic peptide (CNP) is a human C-type natriuretic peptide.

17. The method of claim 15, wherein said natriuretic peptide is a recombinant C-type natriuretic peptide.

18. The method of claim 1, wherein said natriuretic peptide is administered by bolus, infusion or a combination of bolus and infusion.

19. The method of claim 18, wherein said natriuretic peptide is administered by bolus.

20. The method of claim 18, wherein said natriuretic peptide is administered by infusion.

21. The method of claim 18, wherein said natriuretic peptide is administered by a combination of bolus and infusion.

22. The method of claim 18, wherein said natriuretic peptide is administered by bolus and intermittent infusion.

23. The method of claim 20 and 21, wherein said infusion is intermittant.

24. The method of claim 20 and 21, wherein said infusion is serial.

25. The method of claim 20 and 21, wherein said infusion is continuous.

26. The method of claim 19, wherein said natriuretic peptide is administered by bolus at a dose of not less than 2 μg/kg body weight.

27. The method of claim 24, wherein said natriuretic peptide is administered by serial infusion at a dose less than 0.01 μg/kg body weight.

28. The method of claim 23, wherein said natriuretic peptide is administered by intermittent infusion at a dose less than 0.01 μg/kg body weight.

29. The method of claim 1, wherein said natriuretic peptide is administered by intravenous, intramuscular, subcutaneous, intradermal, intrasternal, intraperitoneal or intra-articular injection.

30. The method of claim 1, wherein the said natriuretic peptide is administered prophylactically to said mammal.

31. The method of claim 1, wherein said natriuretic peptide is administered to said mammal for 10 hours.

32. The method of claim 16, wherein said natriuretic peptide is administered to said mammal for 4-6 hours weekly.

33. The method of claim 1, wherein said natriuretic peptide is administered to said mammal at an infusion rate of 0.00125 μg/kg body weight/min to 0.01 μg/kg body weight/min.

34. The method of claim 19, wherein said natriuretic peptide is administered to said at 0.005 μg/kg body weight/min.

35. The method of claim 1, wherein said therapeutically effective dose comprises 0.25 μg/kg body weight to 1.75 μg/kg body weight of said natriuretic peptide.

36. A kit for the treatment of congestive heart failure by administration of a therapeutically effective dose of a natriuretic peptide in combination with a diuretic agent, wherein the natriuretic peptide and the diuretic agent are packaged separately.

37. The kit as claimed in claim 36, wherein said natriuretic peptide is selected from the group consisting of an atrial natriuretic peptide (ANP), a B-type natriuretic peptide (BNP) and a C-type natriuretic peptide (CNP).

38. The kit as claimed in claim 37, wherein said natriuretic peptide is a human B-type natriuretic peptide.

39. The kit as claimed in claim 37, wherein said natriuretic peptide is a recombinant human B-type natriuretic peptide. Compositions and methods are disclosed for administration of synergistic levels of a diuretic and a natriuretic peptide, especially a recombinant form of human BNP, or hBNP. These compositions and methods are useful for the treatment of congestive heart failure.
